SEQUENCE LISTING (1 sequence)

SEQ ID NO: 1 - protein, 13 aa, Artificial Sequence
  stem cell mobilizer
FPHFDLSHGSAQV

(19) United States
(12) Patent Application Publication
(10) Pub. No.: US 2014/0088006 A1
(43) Pub. Date: Mar. 27, 2014

(54) Stem Cell Mobilization and Tissue Repair and Regeneration

(71) Applicant: Wellstat Therapeutics Corporation, Gaithersburg, MD (US)

(72) Inventors: Irena Tsyrlova, Bethesda, MD (US); Fawn Petty, Bowie, MD (US); Reid W. Von Borstel, Potomac, MD (US); Julian Reading, Frederick, MD (US)

(73) Assignee: Wellstat Therapeutics Corporation, Gaithersburg, MD (US)

(21) Appl. No.: 14/118,313

(22) PCT Filed: May 18, 2012
(86) PCT No.: PCT/US2012/038610

§ 371 Date: Nov. 18, 2013

Related U.S. Application Data
(60) Provisional application No. 61/487,548, filed on May 18, 2011.

(51) Int. Cl. C07K 7/08

(52) U.S. Cl. CPC C07K 7/08
USPC 514/6.9; 514/21.5; 514/16.5; 514/13.3

(57) ABSTRACT

Stem cells are mobilized from bone marrow by administering an amount of Phe-Pro-His-Phe-Asp-Leu-Ser-His-Gly-Ser-Ala-Gin-Val (SEQ ID NO: 1) effective to mobilize the stem cells. This method is useful for promoting preservation, repair, or regeneration of bodily tissue, or revascularization, in a patient in need of such treatment. Alternatively, the mobilized stem cells can be collected for transplant.

### BACKGROUND OF THE INVENTION

Adult tissue stem cells including hematopoietic stem cells (HSC) are unique and rare cells responsible for regeneration of different tissues: blood, muscles, hair follicles, skin keratinocytes, pancreatic and neural cells (Orlic et al. 2001, Krause et al. 2001). Stem cell transplantation has been tested in clinical trials for tissue regeneration with a various but low degree of success. This is due to the fact that even after enrichment with the most up-to-date approaches, the resulting HSC populations are not homogeneous. A large proportion of cells may still have no HSC potential, molecular heterogeneity within different HSC subsets and other uncertainties make cell transplantation less feasible for tissue regeneration than HSC mobilization.

Mobilization of stem cells from bone marrow into peripheral blood prior to harvesting is currently being used in clinical settings of allogeneic stem cell transplantation instead of bone marrow. The most common mobilizing agent for clinical uses is granulocyte colony stimulating factor (G-CSF). Other molecules have mobilizing effects on bone marrow cells (AMD3100, IL8, GM-CSF and others), but their effects are indirect and not stem cell specific.

G-CSF, for example, acts on mature bone marrow cells; cells release proteases cleaving the adhesion factors responsible for the retention of cells in bone marrow. The SDF1-CXCR4 axis, important for the retention of cells expressing CXCR4 in the bone marrow, is also involved in the G-CSF effect (Lapidot and Petit 2002). AMD3100, a CXCR4 inhibitor, approved recently for stem cell mobilization induces a more specific mobilization of cells into the circulation than G-CSF via disruption of the CXCR4-SDF1 interaction of bone marrow cells with their microenvironment. However, not only stem cells, but their immature progenitors and even malignant cells in Multiple Myeloma and Acute Promyelocytic leukemia express CXCR4 and therefore migrate into peripheral blood in response to AMD3100 (Kareem, et al. 2009).

Thus, current clinical agents affect multiple cell populations, releasing into circulation high numbers of cells and causing changes in the bone marrow microenvironment. Therefore, these approaches cannot be used for frequent multiple rounds of stem cell mobilization for tissue regeneration.

Under homeostatic conditions many physiological mechanisms including stem cell mobilization are found to be controlled by circadian oscillations; maximal mobilization of HSC into blood stream was found in mice at 5 hr after the onset of light with a reversed circadian HSC mobilization time (early night) demonstrated for human (Lucas, et al. 2008).

HSC can be harvested and expanded or sorted ex vivo for promoting regeneration of tissues, and especially for enhancing revascularization of ischemic tissues. However, this process is expensive and not feasible for repeated use to promote tissue repair or revascularization over time. A preferable approach would be to use a drug that selectively mobilizes endogenous HSC and other repair-promoting progenitors from the bone marrow to enhance or enable tissue repair and revasculaization.

### SUMMARY OF THE INVENTION

This invention provides a method of mobilizing stem cells from bone marrow of a subject, comprising administering to the subject an amount of Phe-Pro-His-Phe-Asp-Leu-Ser-His-Gly-Ser-Ala-Gln-Val (SEQ ID NO: 1) (also referred to as Compound X, or Cpd. X) effective to mobilize the stem cells. This invention provides a compound (SEQ ID NO: 1) for use in mobilizing stem cells from bone marrow of a subject. This invention provides the use of a compound (SEQ ID NO: 1) in the manufacture of a medicament for mobilizing stem cells from bone marrow of a subject. And this invention provides a pharmaceutical composition comprising a compound (SEQ ID NO: 1) for mobilizing stem cells from bone marrow of a subject.

This method, compound, use, and pharmaceutical composition is useful for promoting one or more of preservation, repair, or regeneration of bodily tissue, or revascularization in a patient in need of such treatment. Alternatively, the mobilized stem cells can be collected for transplant.

### BRIEF DESCRIPTION OF THE FIGURES

FIG. 1: Stem cell mobilization after single subcutaneous injection of Compound X.

FIG. 2: Stem cell mobilization after single subcutaneous injection of Compound X or AMD3100.

FIG. 3: CFU-GEMM number in peripheral blood of mice 1 hr after injection of Compound X daily for 4 days.

FIG. 4: CFU-GM number in peripheral blood of mice 1 hr after injection of Compound X daily for 4 days.

FIG. 5: CFU-GM and CFU-GEMM number in peripheral blood of mice injected with G-CSF and Compound X daily for 4 days.

FIG. 6: Cell migration toward SDF1α: CD34+ cells were expanded in presence of Compound X or expanded without Compound X and pulsed for 3 hr with Compound X before the assay

FIG. 7: Cell migration toward SDF1α: effect of AMD3100 in presence or absence of Compound X.

FIG. 8: Survival of STZ treated mice.

FIG. 9: Pancreatic insulin 12 weeks after STZ treatment of C57Bl6 mice.

FIG. 10: Mobilization of CFU-GEMM cells into peripheral blood of diabetic db/db mice after intravenous injection of 5 mg/kg Compound X.

FIG. 11: Wound healing rates in db/db mice treated intravenously with Compound X for 4 days.

FIG. 12: Number of blood vessels in wounds of mice treated with Compound X versus saline.

FIG. 13: Number of blood vessels counted in the wound dermis area on day 10 post-wounding visualized by rabbit anti-α-smooth muscle actin antibodies.

FIG. 14: Glucose level in blood of mice before wound healing study and 7 weeks after the treatment.

FIG. 15: Wound healing rates in db/db mice treated intravenously with Compound X for 4 days.

FIG. 16: Fasting serum glucose in db/db mice treated with Compound X or vehicle for 5 weeks.

### DETAILED DESCRIPTION OF THE INVENTION

Stem cells are mobilized from the bone marrow by a stem cell-stimulatory amount of (SEQ ID NO:1). In humans, the appropriate amount of (SEQ ID NO: 1) is generally from about 100 micrograms to about 10 milligrams per administration, administered from 1 time per week to 3 times per day. For mobilization of stem cells in intensive acute therapy such as shortly after a stroke, from 2 milligrams to 10 milligrams per administration, administered 1, 2, or 3 times per day is preferred. For repeated low dose administration such as for vascular maintenance from 100 micrograms to 1 milligram per administration, administered from once per day to once per week, is preferred. In other embodiments the appropriate amount of (SEQ ID NO: 1) is from about 100 micrograms to about 1 milligram per day for one or more days. For example, the amount can be administered on each of four consecutive days. Typical amounts are, for example, 100 micrograms, 300 micrograms, 500 micrograms, or 1 milligram per day. Thus, in more specific embodiments of this invention, the ranges are from 90 to 110 micrograms, from 280 to 320 micrograms, from 450 to 550 micrograms, or from 900 to 1100 micrograms per day. (If too little is administered, the stem cells may be inhibited rather than stimulated, and mobilization may not occur. Inhibitory doses to be avoided are typically in the range of 50 ng to 1 microgram per day in a human.) Injection, for example intravenous, subcutaneous or intramuscular injection, is a preferred route of administration. Intravenous injection is most preferred for strength of response, and is generally preferred in diabetic patients because of the compromised vasculature in that population. In accordance with this invention, bodily tissues generally can be regenerated or revascularized. In one embodiment pancreatic tissue is regenerated or preserved, particularly insulin-producing islets. In another embodiment dermal tissue is regenerated, for example in wound healing. A general activity that applies to repair or preservation of many different tissues is promotion or enhancement of revascularization. In accordance with this invention, in one embodiment, compounds and methods of the invention are used to promote revascularization of ischemic tissues, including skeletal muscle, heart and brain. In another embodiment, compositions and methods of the invention are used to prevent pancreatic islet failure, e.g. in type 2 diabetes, or to promote islet regeneration in type 1 and type 2 diabetes.

Most end-stage cells in renewing organs are short-lived and must be replaced continuously throughout life. The constant repopulation of renewing organs is driven by a group of undifferentiated cells called stem cells. Stem cells have the unique characteristic of being able to divide and to give rise to more differentiated progenitor cells (“differentiation”) as well as to other stem cells (“self-renewal”). The ability to self-renew ensures that the population of stem cells is not depleted. Rapidly renewing tissues where stem cells have classically been demonstrated include hematopoietic tissue, skin, stomach, intestine, and testes.

Stem cells may be classified according to their differentiation potential as totipotent, pluripotent or multipotent. Totipotent stem cells are capable of forming any tissue in the body. The best example of this is the fertilized egg, which gives rise to both the embryo proper as well as the placenta and supporting tissues. Pluripotent stem cells can form a large subset of body tissues that can include most or all the tissues in the adult whereas multipotent stem cells have a more restricted repertoire of differentiation. Tissue progenitor cells are stem cells that can only differentiate into the cells that constitute a particular type of tissue.

Stem cells can produce new cells to repair damage to tissues and therefore have great potential for regenerative medicine. However, they exist in small quantities in tissues and especially in peripheral blood, making it difficult to collect them or use them clinically. To increase percentage of stem cells and their progenitors in peripheral blood, their mobilization by G-CSF prior to harvest has been used extensively. Mobilized stem cells can repair tissues if their homing and engraftment functions are not impaired (Rafii &Lyden, 2003). There is a need, therefore, to identify compounds that regulate mobilization of pluripotent stem cells and methods of uses for therapeutic purposes. WO 97/36922 (Pro-Neuron, Inc.) and WO 96/10634 (Pro-Neuron, Inc.), both incorporated herein by reference, disclose a tridecapeptide with the sequence Phe-Pro-His-Phe-Asp-Leu-Ser-His-Gly-Ser-Ala-Gln-Val (SEQ ID NO: 1), also referred to herein as ‘Compound X’, that acts on stem cells. Compound X mobilizing activity is now demonstrated and compared to other known mobilizing agents.

The chemokine, stromal cell-derived factor-1 (SDF-1/CXCL12), which binds and activates the CXCR4 receptor, has been implicated as an important mechanism for retention of stem cells within bone marrow microenvironment and mobilization into peripheral blood. AMD3100, a specific antagonist of SDF-1/CXCL12 binding to CXCR4, has been clinically tested and approved for synergizing with granulocyte colony-stimulating factor (G-CSF) to greatly enhance G-CSF-induced mobilization of HSCs/HPCs (Lapidot, T. and I. Petit. 2002, Lapidot et al., 2005).

CD26 is a cell-surface protein, which is a dipeptidylpeptidase IV (DPPIV) and has the capacity to truncate SDF-1/CXCL (De Meester et al., 1999). Human DPPIV is a 110 kDa cell surface molecule it contains intrinsic dipeptidyl peptidase IV activity, which selectively removes N-terminal dipeptide from peptides with proline or alanine in the third amino acid position. It interacts with various extracellular molecules and is also involved in intracellular signal transduction cascades. The multifunctional activities of human DPPIV are dependent on cell type and intracellular or extracellular conditions that influence its role as a proteolytic enzyme, cell surface receptor, co-stimulatory interacting protein and signal transduction mediator. Human DPPIV has a short cytoplasmatic domain from amino acid position 1 to 6, a transmembrane region from amino acid position 7 to 28, and an extracellular domain from amino acid position 29 to 766 with intrinsic DPPIV activity.

DPPIV-deficient mice exhibit resistance to diet-induced obesity, reduced fat accumulation, decreased plasma levels of leptin, increased pain sensitivity, reduced stress-like responses. DPPIV has been implicated in the control of lymphocyte and immune function, cell migration, viral entry, cancer metastasis, and inflammation; deletion of CD26 resulted in decreased mobilization of HPCs in response to exogenous administration of G-CSF (reviewed in Broxmeier et al. 2007). DPPIV also regulates migration of human cord blood CD34+ progenitor cells and the homing and engraftment of hematopoetic stem cells Inhibition of DPP-4 enzymatic activity promotes human hematopoetic stem cell migration and bone marrow engraftment via potentiation of the levels of intact CXCL12/SDF-1α, a physiological substrate for DPP-4 activity (Christopherson e.a 2002 and 2003).

The invention will be better understood by reference to the following examples, which illustrate but do not limit the invention described herein.

### EXAMPLES

### Example 1

### Mobilization of Mouse Stem Cells from Bone Marrow into Peripheral Blood after Single Subcutaneous Injection of Compound X

Stimulatory doses of Compound X activate quiescent stem cells, induce them to proliferate and egress from bone marrow into peripheral blood. This example shows that an increase in number of hematopoietic stem cells (HSC) and their hematopoietic progenitors (HPC) happens within 1 hr after injection of stimulatory doses of Compound X and causes no changes in either cell number or cell composition.

C57Bl6 male mice, from 8 weeks to 4 month of age, from Harlan were used. Mice received subcutaneous injections of saline, Compound X (50 μg/kg) or Compound X (250 μg/kg). After 1 and 4 hours, mice were anesthetized and blood was collected through the orbital sinus with heparinized capillary tubes. 20 μl of blood was removed from each tube for WBC counts;

200 μl of blood was used for FACS analysis of cells phenotypes: briefly 50 μl of blood was transferred to a 15 ml polypropylene tube containing 1 ml ACK lysis buffer and the tubes left at room temperature for 6 minutes. 50 μl of a 1:50 dilution of Fc block (Pharmingen 01241A) along with 2 ul of anti-CD19FITC (Pharmingen 09654A) and 2 μl of anti-Gr-1PE (Pharmingen 01215A) was added and the tubes left at room temperature for 30 minutes protected from light. The cells were washed with 3 ml FACS buffer, resuspended in 300 μl FACS buffer and kept on ice, protected from light until acquired on the FACSCalibur.

Remaining blood from each group was pooled and mononuclear cells were isolated by density gradient separation. To enumerate HSC and HPC in peripheral blood cells were washed, counted plated in semi-solid Methylcellulose MC3434 (Stem cell technology) at 100,000 per dish (5 dishes/group) and placed in CO₂ incubator at 37° C. Colony-forming unit-granulocyte erythroid macrophage megakaryocyte (CFU-GEMM) and Colony-forming unit-granulocyte macrophage (CFU-GM) were detected under inverted microscope 7 and 10 days after. Data presented in Tables 1, 2, and 3, and FIG. 1.

TABLE 1
White blood cell number in peripheral blood of individual
mice 1 hr and 4 hr after subcutaneous injection of
Compound X or Saline

| | White Blood Cell counts: | | | |
|---|---|---|---|---|
| | 1 hour | | 4 hours | |
| | Cell Count | | Cell Count | |
| Sample | (×10⁶/ml) | Volume (μl) | (×10^{6/}ml) | Volume (μl) |
| 1-saline | 10.4 | 600 | 4.2 | 500 |
| 2-saline | 17.1 | 400 | 25.1 | 620 |
| 3-saline | 13.6 | 470 | 18.9 | 550 |
| 4-Cpd. X-50 | 14.1 | 800 | 22.2 | 300 |
| 5-Cpd. X-50 | 7.8 | 700 | 6.9 | 600 |
| 6-Cpd. X-50 | 9.6 | 600 | 10.0 | 700 |
| 7-Cpd. X-250 | 9.6 | 550 | 16.7 | 600 |
| 8-Cpd. X-250 | 9.2 | 700 | 14.6 | 700 |
| 9-Cpd. X-250 | 17.9 | 600 | 10.3 | 700 |

TABLE 2
Number of CFU in peripheral blood of mice at different
time points after single injection of Compound X.

| CFU-GM + | | | |
|---|---|---|---|
| CFU-GEMM | Control-Saline | Cpd. X-50 μg/kg | Cpd. X-250 μg/kg |
| Per 100000 cells | 7 | 4 | 24 |
| 1 hour | 5 | 3 | 28 |
| | 5 | 7 | 29 |
| | 5 | 5 | 24 |
| | 6 | 6 | 20 |
| MEAN | 5.6 | 5 | 25 |
| STD ERR | 0.4 | 0.7 | 1.6 |
| per 100000 cells | 4 | 1 | 1 |
| 4 hour | 5 | 1 | 1 |
| | 4 | 0 | 1 |
| | 6 | 1 | 3 |
| | 4 | 0 | 1 |
| MEAN | 4.6 | 0.6 | 1.4 |
| STD ERR | 0.4 | 0.2 | 0.4 |

White blood cell count differs in different mice but Compound X injection did not cause massive egress of cells from bone marrow into peripheral blood (Table 1). However, stem cell number in blood was increased substantially 1 hr after 250 μg/kg dose of Compound X but not after injection of 50 μg/kg of Compound X. Thus, Compound X was able to increase CFU number in blood very rapidly during 1 hr after injection, the number of HSC HPC increased up to 5 times, but cell flux was transient as demonstrated by low number of CFU found in all groups at 4 hr after Compound X injection.

Different doses of Compound X were tested from 5 μg/kg up to 5 mg with mobilizing activity found only between 125-500 μg/kg of Compound X. Of interest that the Compound X effect on HSC and HPC was not accompanied by mobilization of other cells insofar as cell number and phenotypes of cells in peripheral blood were not affected. Flow cytometry data are presented in Table 3.

TABLE 3
Cell phenotypes in peripheral blood of mice 1 hour and 4 hours
after subcutaneous injection of Compound X.

| | Phenotype | | |
|---|---|---|---|
| | CD19+ | Gr-1 High | Gr-1 Low |
| Sample | | | |
| 1 hr | | | |
| 1: Saline | 36.0% | 14.3% | 3.3% |
| 2: Saline | 40.7% | 16.8% | 5.6% |
| 3: Saline | 46.1% | 17.7% | 4.3% |
| 4: Cpd. X 50 μg/kg | 38.3% | 26.0% | 3.5% |
| 5: Cpd. X 50 μg/kg | 41.0% | 27.7% | 7.0% |
| 6: Cpd. X 50 μg/kg | 29.6% | 29.4% | 3.5% |
| 7: Cpd. X 250 μg/kg | 14.8% | 55.2% | 3.5% |
| 8: Cpd. X 250 μg/kg | 4.8% | 18.7% | 2.1% |
| 9: Cpd. X 250 μg/kg | 14.5% | 6.8% | 2.4% |
| Sample | | | |
| 4 hr | | | |
| 1: Saline | 51.5% | 16.7% | 10.9% |
| 2: Saline | 55.2% | 5.9% | 5.7% |
| 3: Saline | 55.1% | 7.1% | 5.8% |
| 4: Cpd. X 50 | 33.9% | 25.0% | 6.2% |
| 5: Cpd. X 50 | 40.9% | 16.7% | 7.6% |
| 6: Cpd. X 50 | 29.1% | 29.1% | 5.3% |
| 7: Cpd. X 250 | 46.7% | 16.1% | 5.1% |
| 8: Cpd. X 250 | 48.5% | 12.9% | 5.5% |
| 9: Cpd. X 250 | 46.6% | 11.7% | 6.6% |

Thus, one hour after injection Compound X mobilizing effect is fast) and stem cell specific unlike G-CSF or IL8 induced stem cell mobilization caused by an indirect mechanism of protease activation

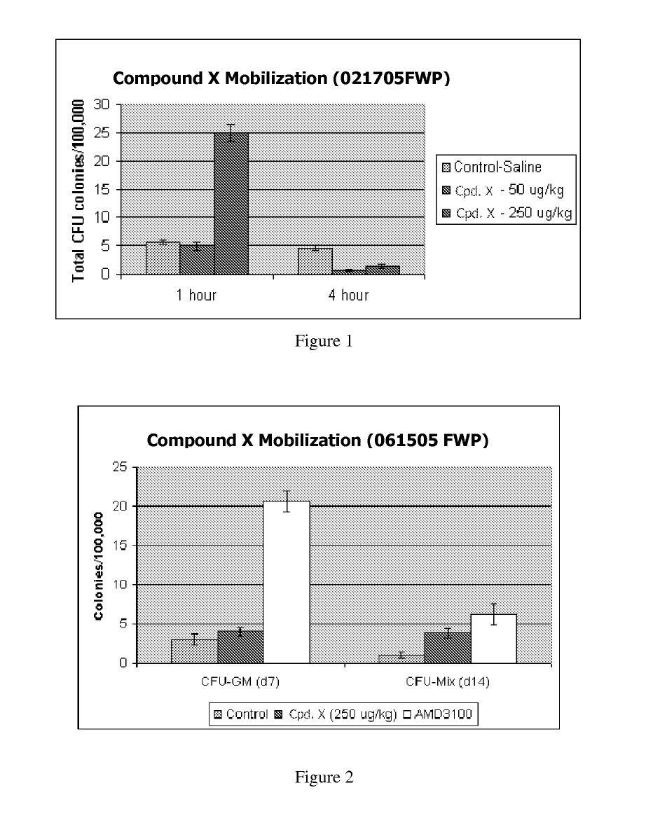

AMD3100 also has fast mobilizing effect and works via Stromal-Derived Factor-1/CXCL12 mediated migration and homing Comparison of mobilizing effect of a single dose of Compound X and an optimal published dose of AMD3100 is presented in Tables 4 and 5, and FIG. 2.

In this experiment, C57Bl6 mice received subcutaneous injections of PBS, Compound X (250 μg/kg) or AMD3100 (5 mg/kg). After 1 hr blood was collected through the orbital sinus with heparinized capillary tubes. Blood from each group was pooled, counted and mononuclear cells were isolated by density gradient separation. Cells were washed, counted and plated at 100,000 per dish (5 dishes/group) for CFU-GEMM and CFU-GM colonies. CFU-GEMM colonies were scored on day 14, while CFU-GM colonies on day 7 after plating in MC3434 (Stem cell Technology).

For flow cytometry analysis the blood was stained with antibodies and a phenotype determined by FACS as described before. In addition to the whole blood FACS phenotyping, Ficolled blood was also stained to monitor the Lin−/Sca+ phenotype. The cells were washed with 3 ml FACS buffer, resuspended in 300 μl FACS buffer and kept on ice, protected from light until acquired on the FACSCalibur

TABLE 4
White blood cell count in peripheral blood of mice 1 hr after
Compound X or AMD3100 subcutaneous injection.

| | Cell Count | |
|---|---|---|
| Sample | (×10⁶/ml) | Volume (ml) |
| PBS | 6.8 | 2.2 |
| Cpd. X-250 μg/kg | 7.2 | 2.1 |
| AMD3100 5 mg/kg | 11.4 | 1.7 |

TABLE 5
Number of CFU-GEMM and CFU-GM in peripheral blood of
mice after single injection of Compound X or AMD3100.

| | Control-PBS | Cpd. X-250 μg/kg | AMD3100-5 mg/kg |
|---|---|---|---|
| CFU-GM (day 7) | | | |
| per 100,000 cells | 2 | 6 | 19 |
| 1 hour | 4 | 4 | 19 |
| | 3 | 4 | 20 |
| | 5 | 3 | 19 |
| | 1 | 3 | 26 |
| MEAN | 3 | 4 | 20.6 |
| STD ERR | 0.71 | 0.55 | 1.36 |
| CFU-GEMM | | | |
| per 100,000 cells | 2 | 6 | 5 |
| 1 hour | 0 | 3 | 4 |
| | 0 | 4 | 11 |
| | 2 | 3 | 7 |
| | 1 | 3 | 4 |
| MEAN | 1 | 3.8 | 6.2 |
| STD ERR | 0.45 | 0.58 | 1.32 |

Contrary to AMD3100, which induced an increase in white blood counts and a substantial increase in number of circulating committed precursors of granulocytes and macrophages—CFU-GM (FIG. 2), the injection of Compound X did not change the number of white blood cells and CFU-GM in peripheral blood of mice. Compound X increased CFU-GEMM number 3.8 times while AMD3100 raised CFU-GEMM in peripheral blood 6.2 times (Table 4; FIG. 2).

It has been known from the literature that AMD 3100 increases the number of early progenitor cells (lineage negative) in peripheral blood within 30 min and 1 hr after mobilization. The results presented in Table 6 show that AMD3100 indeed mobilized up to 3 times more lineage negative progenitors, while Compound X did not have such an effect.

TABLE 6
Phenotype of mononuclear cells from peripheral blood of
mice injected with Compound X or AMD3100.

Phenotype

| | | |
|---|---|---|
| | Lin−/ | |
| Sample | Sca-1− | Lin−/Sca-1+ |

30-min

| | | |
|---|---|---|
| 1: Saline | 1.1% | 0.5% |
| 2: Cpd. X 50 μg/kg | 1.0% | 0.5% |
| 3: Cpd. X 250 μg/kg | 1.1% | 0.6% |
| 4: AMD3100 5 mg/kg | 2.1% | 0.5% |

1-hour

| | | |
|---|---|---|
| 5: Saline | 0.7% | 0.5% |
| 6: Cpd. X 50 μg/kg | 0.8% | 0.4% |
| 7: Cpd. X 250 μg/kg | 1.1% | 0.8% |
| 8: AMD3100 5 mg/kg | 2.8% | 0.6% |

Although both Compound X and AMD3100 are fast mobilizers, they had different effect on bone marrow cells. Contrary to AMD3100, Compound X did not increase mobilization of immature precursor cells (Lin−) and the effect of Compound X was specific to the mobilization of CFU-GEMM and did not induce mobilization of CFU-GM.

### Example 2

### Mobilization of Mouse Stem Cells from Bone Marrow into Peripheral Blood after Multiple Subcutaneous Injection of Compound X

The low levels of circulating HSPC are drastically increased in response to repeated stimulation with the cytokine G-CSF. This example is based on a protocol for a 4-day course of once-daily Compound X injections into C57Bl/6 male mice similar to G-CSF. Two effective dose levels of Compound X were found previously to mobilize HSC 1 hr after single injection and were selected for this experiment. Four-month old C57Bl/6 male mice (Jackson Laboratories) were subcutaneously injected with 0.9% saline, 125 μg/kg Compound X or 250 μg/kg Compound X on days 1, 2, 3 and 4 as described in Table 4. Each day, one-hour post injections, peripheral blood from 3 mice per group was harvested from the orbital plexus into EDTA-containing tubes. On day 4, a femur and spleen were also removed from mice to assess by FACS whether any toxicity aroused from repeated injections of Compound X.

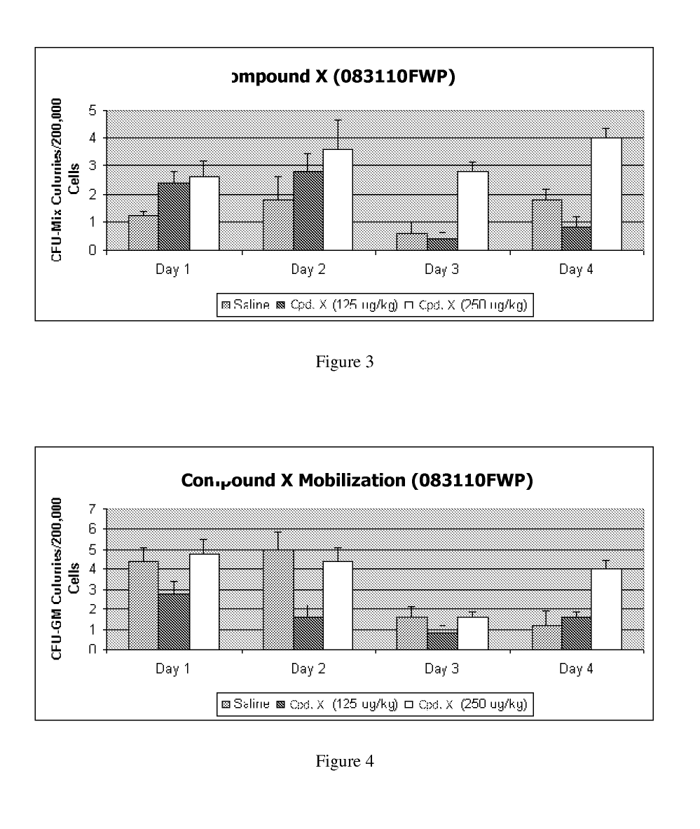

As in the previous example to determine mobilization of colony-forming cells out of the bone marrow, mononuclear cells from peripheral blood were plated into MethoCult 3434 media (Stem Cell Technologies). Briefly, after white blood cell counting, the remaining peripheral blood was diluted (1:1, v/v) with 0.9% saline (APP Pharmaceutical) and layered (2:1, v/v) onto 1-Step™1.077 A (Accurate Chemicals) in 15-ml centrifuge tubes. Cells were centrifuged at 600×g for 20 min at room temperature. The mononuclear cells were harvested from the interface between the plasma layer and the 1-Step™ A solution using a 1-ml syringe and blunt-end needle. The cells were transferred to 15-ml centrifuge tubes and washed twice in 1×PBS. Cells were resuspended in 1 ml of IMDM and cell counts were obtained using the Coulter counter. Cells were placed into 35-mm Petri dishes at 200,000 cells per dish with 5 dishes per group. Cells incubated for 10 days in a 37° C. and 5% CO₂ humidified incubator. CFU colonies were then scored using an inverted microscope—CFU-GM detected on day 7 and CFU-GEMM—on day 10. Data presented in Tables 7, 8 and 9, FIGS. 3 and 4.

Both doses of Compound X mobilized stem cells from bone marrow into peripheral blood 1 hr after subcutaneous injection. White blood cell number did not change after Compound X multiple daily injections.

TABLE 7
Treatment protocol: subcutaneous multiple doses of Compound X injected daily to C57Bl/6 mice.

| N | Treatment Day 1 | N | Treatment Day 2 | N | Treatment Day 3 | N | Treatment Day 4 |
|---|---|---|---|---|---|---|---|
| 12 | Saline | 9 | Saline | 6 | Saline | 3 | Saline |
| 12 | Cpd. X (125 μg/kg) | 9 | Cpd. X (125 μg/kg) | 6 | Cpd. X (125 μg/kg) | 3 | Cpd. X (125 μg/kg) |
| 12 | Cpd. X (250 μg/kg) | 9 | Cpd. X (250 μg/kg) | 6 | Cpd. X (250 μg/kg) | 3 | Cpd. X (250 μg/kg) |

TABLE 8
White blood cell number in peripheral blood of mice 1 hr after
injection of Compound X daily for 4 days.

| Injections: | Day 1 | Day 2 | Day 3 | Day 4 |
|---|---|---|---|---|
| Saline | 7.8 | 8.4 | 8.6 | 9.8 |
| Cpd. X (125 μg/kg) | 7.4 | 8.8 | 6.0 | 8.6 |
| Cpd. X (250 μg/kg) | 8.6 | 8.2 | 10.2 | 9.2 |

TABLE 9
CFU-GEMM number per 100000 mononuclear cells in peripheral blood of
mice 1 hr after injection of Compound X daily for 4 days.

| | | | | | | AVG | STD ERR |
|---|---|---|---|---|---|---|---|
| Day 1 | | | | | | | |
| Saline | 1 | 2 | 1 | 1 | 1 | 1.2 | 0.20 |
| Cpd. X (125 μg/kg) | 2 | 2 | 4 | 2 | 2 | 2.4 | 0.40 |
| Cpd. X (250 μg/kg) | 4 | 1 | 2 | 2 | 4 | 2.6 | 0.60 |
| Day 2 | | | | | | | |
| Saline | 1 | 1 | 1 | 1 | 5 | 1.8 | 0.80 |
| Cpd. X (125 μg/kg) | 5 | 3 | 1 | 2 | 3 | 2.8 | 0.66 |
| Cpd. X (250 μg/kg) | 2 | 6 | 3 | 6 | 1 | 3.6 | 1.03 |
| Day 3 | | | | | | | |
| Saline | 0 | 1 | 2 | 0 | 0 | 0.6 | 0.40 |
| Cpd. X (125 μg/kg) | 1 | 0 | 0 | 1 | 0 | 0.4 | 0.24 |
| Cpd. X (250 μg/kg) | 2 | 4 | 2 | 3 | 3 | 2.8 | 0.37 |
| Day 4 | | | | | | | |
| Saline | 2 | 3 | 1 | 1 | 2 | 1.8 | 0.37 |
| Cpd. X (125 μg/kg) | 0 | 2 | 1 | 0 | 1 | 0.8 | 0.37 |
| Cpd. X (250 μg/kg) | 4 | 3 | 5 | 4 | 4 | 4 | 0.32 |

As compared to vehicle control (saline), the administration of Compound X (250 μg/kg) over 4 days resulted in the consistent mobilization of progenitor cells from the peripheral blood. A 2-fold increase or more was seen in the multilineage CFU-GEMM colonies on all days with day 3 producing a 4.7-fold increase in CFU-GEMM. Compound X like in the previous examples did not affect mobilization of granulocytic and macrophage precursors CFU-GM.

### Example 3

### Combined Effect of Multiple Subcutaneous Injections of Compound X with G-CSF Mobilizing HSC and HPC into Peripheral Blood

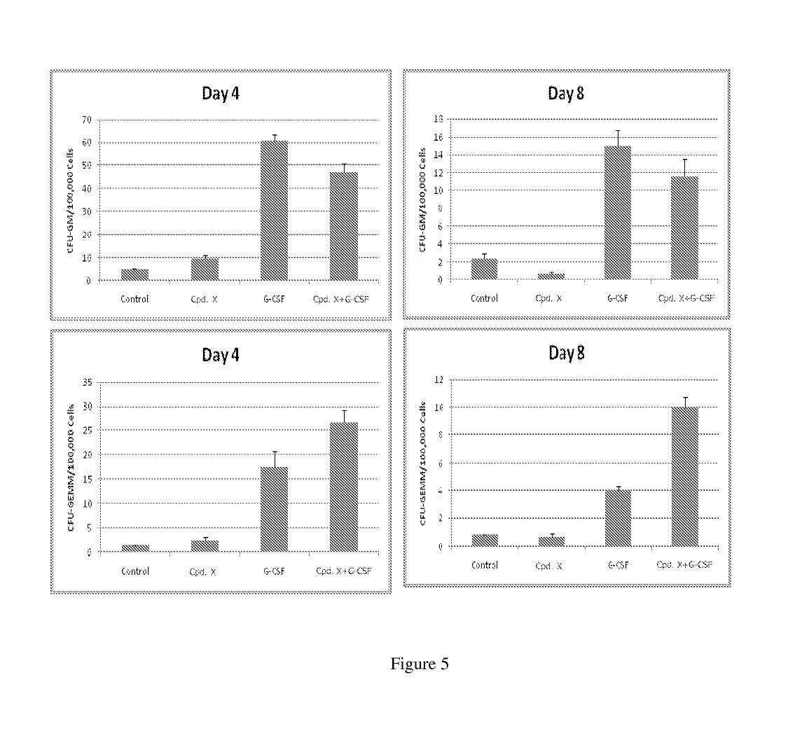

Compound X and Neupogen (rG-CSF Amgen, lot# P043601) were diluted in PBS. Mice received subcutaneous injections of PBS, Compound X (5 μg/mouse), G-CSF (5 μg/mouse) or Compound X (5 μg/mouse)+G-CSF (5 μg/mouse) for four consecutive days. On the last day of injections, 1 hour post injections, blood was collected through the orbital sinus with heparinized capillary tubes. Blood was pooled from 3 mice/group and white blood cell counts were obtained. Mononuclear cells were isolated by density gradient separation from pooled blood. Cells were washed, counted and plated at 100,000 per dish (5 dishes/group) for CFU-mix and CFU-GM colonies. Blood was also collected and processed on day 8 for colony assay. White blood cell count is presented in Table 10. Results are shown in Tables 10, 11, and 12 and in FIG. 5.

TABLE 10
White blood cell count in peripheral blood of mice injected
with G-CSF and Compound X daily for 4 days.

| | | |
|---|---|---|
| | Cell Count | |
| Sample | ×10⁶ | Volume (ml) |

White Blood Cell counts: (Day 4)

| | | |
|---|---|---|
| Control (PBS) | 8.8 | 1.9 |
| Compound X | 7.3 | 1.8 |
| G-CSF | 14.6 | 1.8 |
| Compound X + G- | 16.4 | 1.8 |
| CSF | | |

White Blood Cell counts: (Day 8)

| | | |
|---|---|---|
| Control (PBS) | 7.4 | 1.8 |
| Cpd. X | 6.1 | 1.5 |
| G-CSF | 5.0 | 1.9 |
| Cpd. X + G-CSF | 6.4 | 2.2 |

TABLE 11
CFU-GM numbers per 100000 mononuclear cells in peripheral
blood of mice injected daily with G-CSF and Compound X for
4 days.

| | Control-PBS | Cpd. X | G-CSF | Cpd. X + G-CSF |
|---|---|---|---|---|
| Day 4 | | | | |
| | 5 | 11 | 52 | 52 |
| | 7 | 11 | 68 | 55 |
| | 4 | 7 | 62 | 50 |
| | 5 | 6 | 58 | 36 |
| | 4 | 13 | 63 | 43 |
| MEAN | 5 | 9.6 | 60.6 | 47.2 |
| STD | 0.55 | 1.33 | 2.68 | 3.43 |
| ERR | | | | |
| Day 8 | | | | |
| | 4 | 1 | 12 | 18 |
| | 2 | 0 | 15 | 12 |
| | 3 | 0 | 11 | 7 |
| | 1 | 1 | 21 | 13 |
| | 2 | 1 | 16 | 8 |
| MEAN | 2.4 | 0.6 | 15.0 | 11.6 |
| STD | 0.51 | 0.24 | 1.76 | 1.96 |
| ERR | | | | |

TABLE 12
CFU-GEMM numbers per 100000 mononuclear cells in
peripheral blood of mice injected daily with G-CSF and
Compound X for 4 days.

| | Control- | | | |
|---|---|---|---|---|
| | Saline | Cpd. X | G-CSF | Cpd. X + G-CSF |
| Day 4 | | | | |
| | 3 | 2 | 29 | 32 |
| | 0 | 3 | 20 | 23 |
| | 2 | 3 | 12 | 18 |
| | 0 | 0 | 12 | 31 |
| | 2 | 4 | 15 | 29 |
| MEAN | 1.4 | 2.4 | 17.6 | 26.6 |
| STD | 0.060 | 0.678 | 3.2 | 2.66 |
| ERR | | | | |
| Day 8 | | | | |
| | 0 | 1 | 5 | 12 |
| | 1 | 1 | 4 | 8 |
| | 1 | 1 | 3 | 11 |
| | 1 | 0 | 4 | 10 |
| | 1 | 0 | 4 | 9 |
| MEAN | 0.8 | 0.6 | 4 | 10.0 |
| | 0.02 | 0.245 | 0.316 | 0.707 |

TABLE 13
Fold increase in number of CFU-GEMM and CFU-GM
progenitors after multiple injections of G-CSF and
Compound X.

| | CFU-GM | CFU-GM | CFU-Mix | CFU-Mix |
|---|---|---|---|---|
| Treatment | Day 4 | Day 8 | Day 4 | Day 8 |
| Cpd. X | 1.9 | 0 | 1.7 | 0 |
| G-CSF | 12 | 6 | 12.5 | 5 |
| Cpd. X + G-CSF | 9 | 4.8 | 19 | 12.5 |

G-CSF mobilized many different hematopoietic progenitors similar to the literature results, even after 8 days from the first subcutaneous injection the number of circulating

HSC and HPC remained high (Tables 11 and 12; FIG. 5). Injections of Compound X selectively increased only CFU-GEMM number as measured 1 hr after the last injection 1.7 times; there was no difference found at day 8 in number of CFU-GEMM or CFU-GM. Compound X injected into mice that received G-CSF changed the effect of G-CSF by increasing proportion of immature CFU-GEMM and decreasing CFU-GM (Tables 11 and 12; FIG. 5).

In an adult mouse, HSCs disappear from circulation 1-5 minutes after transplantation, demonstrating extremely rapid homing to target hematopoietic niches; all currently used mobilizers induce long lasting and excessive presence of different myeloid cell types in peripheral blood, this can cause different side effects especially during repeated courses of mobilization. Compound X acting fast and reversibly stimulates proliferation of HSC and their egress from bone marrow bringing stem cell number within 1 hr after injection from a total of approximately 100 HSC (found in circulation during normal homeostatic conditions) up to 300-400 without otherwise changing cell composition in peripheral blood. When used in combination, AMD3100 and G-CSF worked together to generate even higher increases in the number of different types of cells in the bloodstream than each mobilizer alone (Broxmeyer et al. 2005), although it may be useful for harvesting cells it cannot be applied for tissue regeneration involving repeated treatments.

G-CSF was shown to induce stem cell mobilization by up-regulating CXCR4 and decreasing bone marrow SDF1α; AMD3100 works via CXCR4 inhibition, thus, both G-CSF and AMD3100 (plerixafor) mobilize stem cells by disrupting the SDF1/CXCR4 axis (Lapidot and Petit, 2002). The SDF1/CXCR4 axis was shown to have primary importance for stem cell interaction with hematopoietic niche including homing and retention of stem cells. Combined effect of Compound X with G-CSF on stem cell mobilization was not synergistic like the effect of G-CSF with AMD3100, rather Compound X increased proportion of HSC among mobilized by G-CSF myelocytes, demonstrating a different mechanism of action.

### Example 4

### Migration of Human CD34⁺ Cells Toward SDF1α and Effect of Compound X on SDF1 Induced Mobilization

SDF-1-induced migration of CD34⁺ cells in vitro was shown to correlate with hematopoietic recovery after clinical transplantation (Tavor et al. 2005); it was important to test if Compound X affects stem cell motility. CD34⁺ cell migration toward 100 ng/mL SDF-1α in a Corning Transwell device (24 well format, 5 μm pore size, polycarbonate membrane) was studied with and without Compound X and in the presence or absence of either anti-CXCR4 antibody or the CXCR4 antagonist AMD3100. This example shows that Compound X had no effect on SDF1α/CXCR4 interaction.

The CD34⁺ population of cells from human umbilical cord blood was obtained from Lonza, defrosted and expanded ex vivo with cytokine cocktail for 6 days according to standard procedures. Briefly, 10 wells of two 12 well dishes were seeded at a density of 8×10⁴ cells per well in 2 mL of CellGenix serum free growth medium (SCGM). Cells were allowed to expand in a 37° C. incubator (5% CO2, 86% RH) for 6 days. During the expansion, the 10 wells of one of the two plates received treatments of Compound X to a final concentration of 5 ng/mL (10 μL from freshly prepared 1 μg/mL stock in SCGM) at 24 hours and 72 hours after initiation of the culture.

3 hours prior to the start of the migration assay, both control and Compound X expanded cells were pulsed with either 0, 5 ng/mL or 5 μg/mL Compound X. Migration assay was performed according to the method described (Tavor et al. 2005). Cells were centrifuged for 5 minutes at 1100 RPM and resuspended at a density of 2×10⁶ cells/mL in CellGenix SCGM serum free medium containing 1% Fraction V BSA either in the presence or absence of 5 ng/mL or 5 μg/mL Compound X for 3 hours. The feeder tray (bottom) was prepared with 600 μL of serum free SCGM supplemented with 1% BSA in the presence or absence of SDF-1α. All treatments were conducted in triplicate. The wells of the migration chamber (top) received 100 μL of cells from the 2×10⁶ cells/mL suspensions, preincubated with 5 ng/mL or 5 μg/mL Compound X or Control. All treatments were conducted in triplicates. The plates were placed in a 37° C. incubator (5% CO₂, 86% RH) and cells were allowed to migrate for 4 hours. After the incubation the Transwell inserts were removed and discarded from the chamber. The cells that migrated to the lower chamber were transferred to microcentrifuge tubes and were centrifuged for 5 min at 10,000 RPM. The supernatants were aspirated away, and the tubes frozen on dry ice to facilitate cell lysis in the subsequent step.

The cells were thawed at room temperature. Meanwhile, cell lysis buffer/dye solution was prepared fresh by mixing CyQuant GR dye (400× stock) with 1× lysis buffer. 200 μL of the dye/lysis solution was added to the tubes containing the migrated cells, vortexed and incubated 15 minutes at room temperature (dark). 150 μL of the resulting cell lysates were transferred to a new 96 well plate and the fluorescence was read on a Molecular Dynamics SpectraMax M2 fluorimeter (ex 485, em 538). Results are presented in

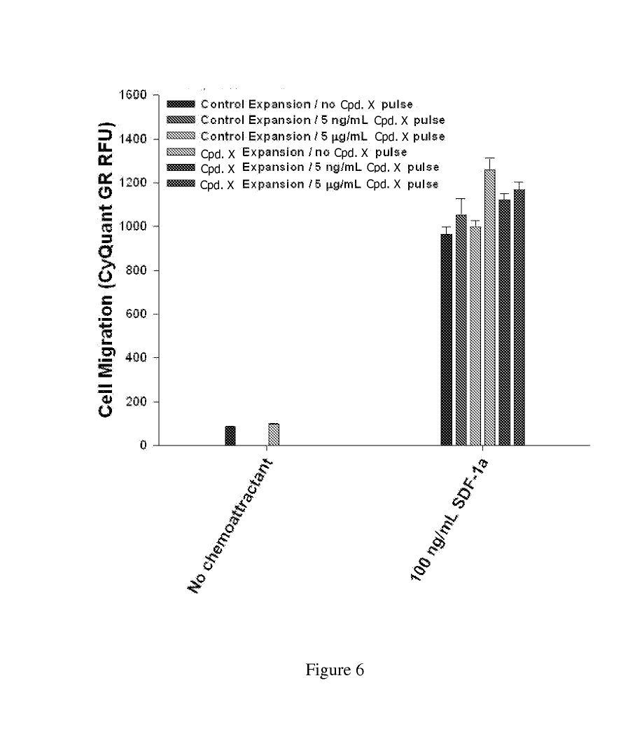

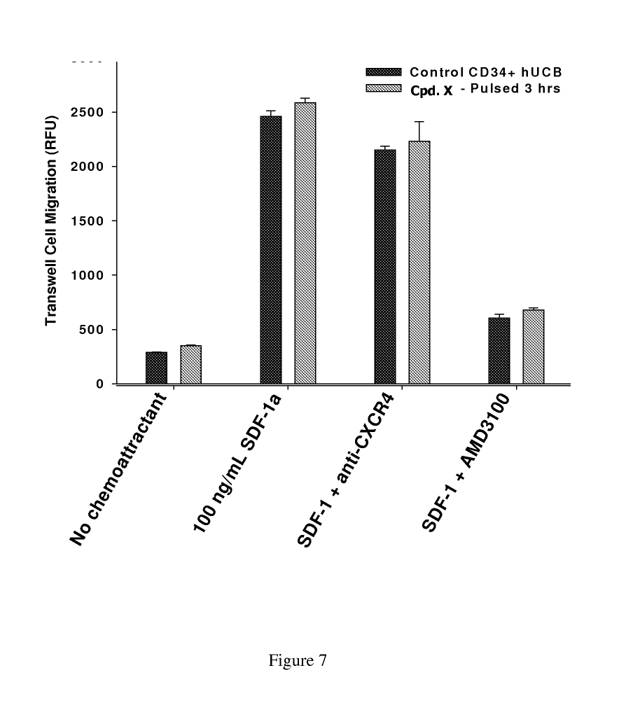

### FIG. 6.

In another experiment (FIG. 7) cells were again expanded with/without a 5 ng/ml dose of Compound X for 6 days and tested for their migratory activity toward SDF1α; effect of pulse with Compound X, anti-CXCR4 antibody or AMD3100 was studied as well (FIG. 7).

Control and Compound X treated cells were pooled separately into two tubes, centrifuged for 5 minutes at 1100 RPM and resuspended at a density of 2×10⁶ cells/mL in CellGenix SCGM serum free medium containing 1% Fraction V BSA, either in the presence or absence of 5 ng/mL Compound X. Compound X treated cells were additionally pulsed with Compound X for 3 hr before the assay. The feeder tray (bottom) was prepared with 600 μL of serum free SCGM supplemented with 1% BSA in the presence or absence of 100 ng/mL SDF-1α. The migration chamber (top) received 100 μL of cells from the 2×10⁶ cells/mL suspension, in the presence or absence of 5 ng/mL Compound X, 10 μg/mL antibodies to CXCR4 or 10 μg/mL AMD3100, as indicated. For the anti-CXCR4 and AMD3100 treatments, 500 μL aliquots of control or Compound X pulsed cells were treated with 5 μL of anti-CXCR4 (1 mg/mL in PBS) or 5 μL of AMD3100 (1 mg/mL in SCGM) prior to adding cells to the migration chamber.

The plate was placed in a 37° C. incubator (5% CO₂, 86% RH) and cells were allowed to migrate for 4 hours. The Transwell inserts were removed and discarded from the chamber after 4 hours migration. The cells that migrated to the lower chamber were transferred to microcentrifuge tubes and were centrifuged for 5 min at 10,000 RPM. The supernatants were aspirated away, and the tubes frozen at −70° C. to facilitate cell lysis in the subsequent step.

The cells were thawed at room temperature. Meanwhile, cell lysis buffer/dye solution was prepared fresh by mixing CyQuant GR dye (400× stock) with 1× lysis buffer. 200 μL of the dye/lysis solution was added to the tubes containing the migrated cells and incubated 15 minutes at room temperature (dark); 150 μL of the resulting cell lysates were transferred to a new 96 well plate and the fluorescence was read on a Molecular Dynamics SpectraMax M2 fluorimeter (ex 485, em 538).

Both the control human umbilical cord CD34⁺ cells and those pulsed for 3 hours with Compound X exhibited robust Transwell migration in this assay (FIG. 7). There was no significant difference in the migration of the control and the Compound X treated cells.

Both control and Compound X treated cells showed only a modest decrease in migration when exposed to 10 μg/mL anti-CXCR4 antibody, but showed strong inhibition when treated with the CXCR4 antagonist AMD3100 (10 μg/mL). Compound X had no effect on stem cell transwell migration toward SDF1, while AMD3100 inhibited stem cell migration substantially. Compound X presence did not interfere with the effect of AMD3100 or anti CXCR4 antibody. SDF1α-CXCR4 axis is not involved in mobilizing effect of Compound X.

### Example 5

### STZ Diabetes Model

In order to look at the ability of Compound X mobilized stem cells to mediate tissue regeneration in vivo subcutaneous injections of 250 μg/kg of Compound X were compared with the ability of Compound X treated bone marrow cells to regenerate pancreas in a streptozoticin-induced diabetic model. Streptozotocin (STZ) is an antibiotic that preferentially kills the insulin secreting beta cells in pancreas. STZ-induced diabetes is a widely used model of pancreatic insufficiency.

Streptozotocin was dissolved and diluted into citric acid buffer (pH 4.5) immediately before injections. Drinking water and food were removed for 6 hours from 8-week old C57BL/6 female mice (Charles River) before mice were given a single intraperitoneal dose of 150 mg/kg STZ (Day −4). Blood for glucose determination was obtained from the tail vein four days post STZ. Blood glucose was measured by using an Elite glucometer (Bayer). Mice were considered diabetic if their blood glucose was >300 mg/dL. Mice showing >300 mg/dL blood glucose were randomized and divided into 5 groups of 10 mice (day 0).

After randomization, STZ-induced diabetic mice STZ-induced diabetic mice received either one expanded bone marrow cell transplants on days +1 or three expanded bone marrow cell transplants on days +1, +2 and +4. To test the effects of Compound X induced mobilization on diabetes in in vivo, one group of diabetic mice was treated with subcutaneous injections of 250 μg/kg of Compound X for 5 consecutive days for 3 weeks while Control group of mice received simultaneous Saline injections. One group of diabetic mice was left untreated.

To prepare cells for injection, 8-week old C57BL/6 male mice (Charles River) were sacrificed and the cells removed from the femurs by flushing with Dulbecco's phosphate buffered saline (PBS). The cells were pelleted by centrifugation and the cells were prepared for density centrifugation using a Percoll gradient. Low density fraction of cells was collected after centrifugation and resuspended in cIMDM containing 2 units/mL human erythropoietin, 100 ng/mL murine interleukin 3 and 100 ng/mL murine stem cell factor (all from R&D Systems) for ex vivo expansion. 2 ml aliquots of 4×10⁵ cells/mL were transferred to the wells of 24-well dishes in 2 mL. Cells were treated with Compound X (500 ng/mL) at 24 and 72 hours.

After 96 hours of expansion, cells were harvested and processed for fluorescence activated cell sorting (FACS). The cell sorting was performed on a FACSVantage SE (BD Biosciences) equipped with an Enterprise II laser and a 70 mm nozzle tip. The alignment and compensation of the FACS was initially checked with CaliBRITE calibration beads (BD Biosciences) according to the manufacturer's instructions. Stained cells were excited with laser 2 (488 nm) and fluorescence emission was detected using the following optical filters, FL-3 (PerCP-Cy5.5) 695/BP40, FL-2 (phycoerythrin) 585/BP42 and FL-1 (FITC) 530/BP30. Each sample was resuspended in complete IMDM (90% IMDM, 10% horse serum), split into four, 2 mL aliquots and transferred to 12 mL polypropylene round-bottomed tubes. 10 μL of CD38FITC, 10 μL of CD49e/VLA-5PE and 10 μL of CD11bPerCP-Cy5.5 (all from Pharmingen) were added to each tube, the cells mixed and kept on ice, protected from light for 15 min. Eight mL cIMDM was added, the cells pelleted by centrifugation for 5 min and supernatant discarded. Cells were resuspended in 1 mL cIMDM (approximately 10×10⁶ cells/mL), passed through a 40 mm filter (Falcon), transferred to 5 ml FACS tubes and kept on ice protected from light until sorted. Cells with a CD381o/VLA-5⁺/CD11b⁻ phenotype (also called ‘R9 cells’) were sorted into cIMDM, pelleted by centrifugation, resuspended in IMDM and a cell count using Trypan Blue (Sigma) exclusion for viability assessment performed on a hemacytometer. Three consecutive ex vivo expansions and R9 sorting were performed in order to transplant mice 3 times.

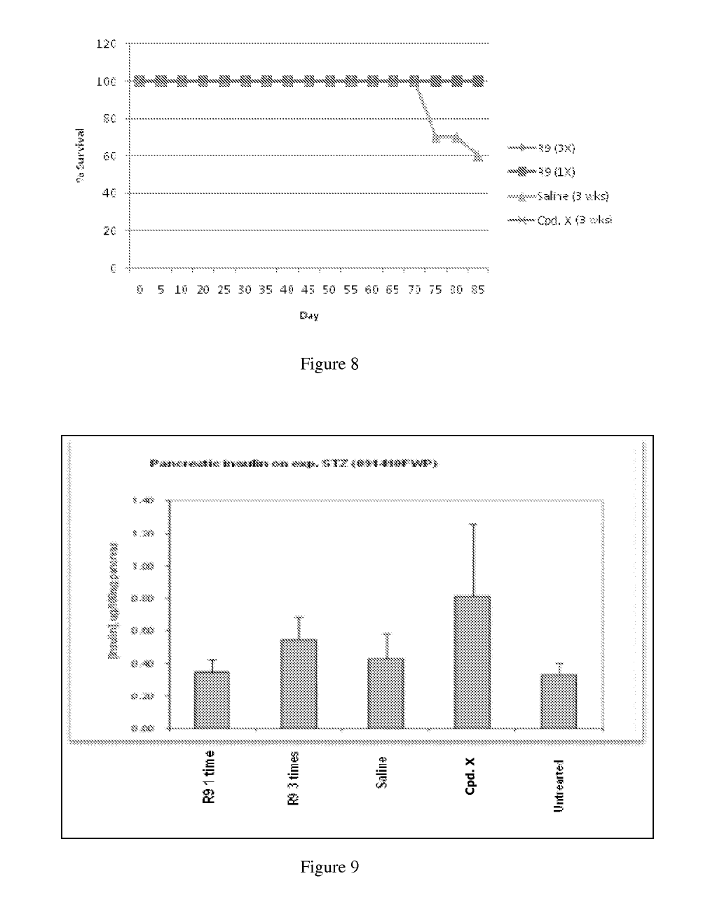

Blood glucose levels and body weight was measured weekly. After 12 weeks, mice were sacrificed, pancreata removed and weighed. One half of pancreas was frozen for histological analysis and the remaining half was frozen for pancreatic insulin analysis. For insulin measurements by Electrochemiluminescence (ECL) assay the tissue was placed in extraction solution (75% ethanol, 0.15N HCl (1N HCl:H2O=30:6.4, 1 ml/60 mg tissue) chopped into very small pieces and sonicated (4 times, 15 seconds each). The sonicator probe was washed between each sample with distilled water and ethanol. The tubes were always placed on ice, and care was taken to prevent the solution from warming up during sonication. The samples were left at −20° C. overnight. Next day, each sample was vortexed thoroughly and centrifuged at 2500 rpm for 5 min The supernatant was separated into 1.5 ml ependorff tubes and centrifuged at 4° C. for 20 min at 1300 rpm. ECL was performed with Bio-anti-rat insulin mAb: 1450 μg/ml in stock (In house labeled Biogenesis Clone 5E4/3) Taq-anti rat insulin mAb: 800 μg/ml in stock (In house labeled Biogenesis Clone 5B6/6), Dynabeads M-280 Streptavidin: 10 mg/ml. ECL measurement of insulin was done using ECL microplate reader. Results are presented in FIG. 8 and FIG. 9.

In other experiments transplantation of R9 cells improved survival of diabetic mice after STZ injection. Dose 150 mg/kg used in this experiment provided longer survival of mice than previously used dose 160 mg/kg. However as shown in FIG. 8 Control mice injected with Saline for 3 weeks began to die while mice injected with stem cell mobilizing doses of Compound X and mice transplanted with R9 cells either one time or three times have survived 100%.

The dose of STZ in this experiment was lower than previously used and did not cause severe cachexia in mice with diabetes, not all mice demonstrated substantial body weight loss: 50% of mice in control STZ group left without treatment and 50% mice in control group treated with Saline demonstrated substantial weight loss (about 20% of their initial body weight). Such weight loss was registered only in 20% mice (2 out of 10) in groups transplanted with R9 cells 1 time or three times. No mice injected with stem cell mobilizing doses of Compound X had any substantial weight loss (data not shown).

Pancreatic insulin was measured by ECL assay as described above. In this model the control insulin levels in female C57Bl/6 mice was found to be 5.768±0.12 μg/100 mg tissue; this level usually dropped to 0.298±0.182 μg/100 mg tissue one week after STZ injection. The results are presented in FIG. 9.

### Example 6

### Multiple Subcutaneous Injections of Compound X Maintain an Increased Level of CFU-GEMM in Peripheral Blood of Mice

Mobilization of hematopoietic stem and progenitor cells from bone marrow into peripheral blood is important for tissue regeneration. However, this physiological mechanism is often insufficient, including in elderly patients and people with diabetes. In order to look at the ability of Compound X mobilized stem cells to mediate tissue regeneration, multiple in vivo injections of Compound X were administered. A group of DBA/2J male mice were injected subcutaneously for 5 consecutive days with 250 ug/kg Compound X in Saline. Another group of mice received injections of vehicle only. Peripheral blood from 3 mice from each of these groups was then harvested from the orbital plexus at 7 days post initial injection. Blood was also harvested and pooled from 3 naïve mice to serve as an additional control group. To determine mobilization of pluripotent colony-forming cells from bone marrow into peripheral blood, mononuclear cells obtained from blood were plated into MethoCult 3434 media (Stem Cell Technologies 03434). Briefly, peripheral blood from three mice was pooled, diluted (1:1, v/v) with 0.9% saline (APP Pharmaceutical) and layered (2:1, v/v) onto 1-Step™1.077 A (Accurate Chemicals AN224510) in 15-ml centrifuge tubes. Cells were centrifuged at 600×g for 20 min at room temperature. The mononuclear cells were harvested from the interface between the plasma layer and the 1-Step™ A solution using a 1-ml syringe and 16G blunt-end needle (Stem Cell Technologies 28120). The cells were transferred to 15-ml centrifuge tubes and washed twice in 1×PBS. The cells were placed into 35-mm Petri dishes at 100,000 cells per dish with 5 dishes per group. Cells incubated for 10 days in a 37° C. and 5% CO2 humidified incubator. CFU-GM colonies consisting of granulocytic and monocytic cells and CFU-GEMM colonies representing mixed cell populations of granulocytes, macrophages and erythroid cells were then scored using an inverted microscope. Blood was taken at Zeitgeber Time 4 (ZT4) which corresponded to the physiological time of cell emigration from bone marrow—the circadian stem cell mobilization peak.

Results are presented in Tables 14 and 15. As shown, injections of Compound X at a dose of 250 ug/kg/day for 5 days did not change the number of circulating CFU-GM, while the total CFU-GEMM number in circulation was 3 times higher on day 7 after the first injection than in naïve mice or mice injected with vehicle.

TABLE 14
CFU-GEMM and CFU-GM number per 100000 mononuclear cells obtained
from peripheral blood of mice on day 7^{th} after the first Compound X or vehicle injection.
CFU colonies/100,000 MNCs

| | CFU-GM | | | | | | | CFU-GEMM | | | | | | |
|---|---|---|---|---|---|---|---|---|---|---|---|---|---|---|
| Group | Colony # | | | | | AVG | SE | Colony # | | | | | AVG | SE |
| Naïve Control | 10 | 5 | 10 | 4 | 4 | 6.6 | 1.40 | 0 | 1 | 3 | 5 | 2 | 2.2 | 0.86 |
| Vehicle Control | 8 | 6 | 7 | 9 | 10 | 8 | 0.71 | 3 | 5 | 3 | 3 | 2 | 3.2 | 0.49 |
| Compound X | 13 | 14 | 13 | 14 | 8 | 12.4 | 1.12 | 12 | 16 | 8 | 8 | 13 | 11.4 | 1.54 |

TABLE 15
CFU-GEMM and CFU-GM number per ml of blood obtained
from peripheral blood of mice on day 7^{th} after the first
Compound X or vehicle injection.
CFU Colonies/ml of Peripheral Blood

| | CFU-GM | | CFU-GEMM | |
|---|---|---|---|---|
| Group | Average | STD ERR | Average | STD ERR |
| Naïve Control | 62.7 | 13.30 | 20.9 | 8.17 |
| Vehicle Control | 60 | 5.30 | 24 | 3.67 |
| Cpd. X (250 ug/kg) | 86.8 | 7.86 | 79.8 | 10.75 |

No accumulation of cells with stem cell phenotypes were found in the spleens of mice treated with Compound X (Table 16). Thus, multiple subcutaneous injections of Compound X were able to induce mobilization of CFU-GEMM that continued for several days after the last injection of peptide without increasing the mobilization of more mature CFU-GM progenitors.

TABLE 16
Flow cytometry analysis of primitive stem cells (Lin−kit+Sca+), self-
renewing stem cells *CD150+CD48− and endothelial precursors (CD34+VEGF−
R2/Flk1+) in spleens of mice on day 7^{th} after the first Compound X or vehicle injection.

| | | Lin− | Lin− | Lin− | | | | | |
|---|---|---|---|---|---|---|---|---|---|
| | | Kit+ | Kit^{low} | Kit+ | | | | | |
| | | Sca- | Sca- | Sca- | CD150+ | CD244+ | CD34+ | CD34+ | CD34− |
| Treatment | Stats | 1+ | 1+ | 1− | CD48− | CD48+ | Flk-1+ | Flk-1− | Flk-1+ |
| A: Cpd. X | Mean | 0.07 | 0.17 | 0.50 | 0.17 | 0.78 | 0.03 | 0.05 | 0.38 |
| (250 μg/kg) | SE | 0.01 | 0.01 | 0.05 | 0.02 | 0.10 | 0.01 | 0.00 | 0.10 |
| B: Saline | Mean | 0.04 | 0.13 | 0.39 | 0.20 | 0.61 | 0.03 | 0.05 | 0.27 |
| control | SE | 0.00 | 0.02 | 0.03 | 0.09 | 0.06 | 0.00 | 0.00 | 0.01 |
| D: Naïve | Mean | 0.06 | 0.15 | 0.47 | 0.22 | 0.68 | 0.03 | 0.05 | 0.29 |
| | SE | 0.00 | 0.01 | 0.00 | 0.03 | 0.03 | 0.00 | 0.00 | 0.02 |

In adult mice, hematopoietic stem cells (HSCs) disappear from circulation 1-5 minutes after transplantation due to extremely rapid homing to target hematopoietic niches. The continued presence of a range of myeloid precursors including CFU-GM in the peripheral blood after G-CSF treatment (and to a lesser extent AMD3100) is therefore far from the physiological condition and may cause side effects during mobilization in healthy donors and patients.

Repeated subcutaneous injection of Compound X into healthy DBA2 mice or C57BL6 mice (data not shown) mobilized activated pluripotent colony-forming stem cells without apparent changes in total cell number or mobilization of CFU-GM progenitors. These data indicate that this reversible mobilizer, Compound X, should avoid the negative features of other mobilizing agents and mimic the physiological mechanism of stem cell mobilization that correlates with the circadian rhythm.

Compound X reversibly stimulates proliferation of HSC and by increasing their egress from bone marrow can bring into circulation double or triple that number of stem cells needed for regeneration without changing the cell composition in either the peripheral blood or the spleen. In a clinical setting this procedure could be repeated every 12 hr generating multiple pulses of physiologically appropriate stem cells for potential tissue regeneration and the prevention of tissue damage associated with aging.

### Example 7

### Multiple Intravenous Injections of Compound X Induce Mobilization of CFU-GEMM in Diabetic db/db Mice and Accelerate Wound Repair

Wound healing and tissue revascularization involves not only local repair processes but also support from bone marrow-derived repair cells. After wounding, a release of endogenous bone marrow progenitor cells was detected first in the peripheral blood then at the wound site in the early stages of wound repair. The level of these released/mobilized progenitor cells in the peripheral blood was reduced in diabetic mice (Fiorina e.a. 2010 and Tepper e.a. 2010). There is considerable variation in the ability of healthy donors to mobilize stem cells and their progenitors; Ferraro et. al. noticed that patients in whom hematopoietic stem cells (HSCs) failed to mobilize had elevated blood glucose and glycated hemoglobin. In addition they showed impaired G-CSF induced mobilization in a mouse model of streptozotocin-induced diabetes, suggesting that there may be a defect in the trafficking of HSCs out of the bone marrow and into the peripheral-blood system in diabetics.

Leptin receptor-deficient diabetic db/db mice are used routinely for studying diabetic wound healing and were selected for the present study. Compound X subcutaneous administration was shown previously to increase efflux of multipotential colony forming progenitors, CFU-GEMM, in healthy mice. Therefore, this study was conducted in order to determine if Compound X could increase mobilization of CFU-GEMM into the circulation and accelerate wound healing in db/db diabetic mice.

Diabetic db/db—BKS.Cg-Dock7m+/+Leprdb/J male mice (Jackson Laboratories, stock #000642) with verified blood glucose levels over 350 mg/dL were used for mobilization experiments. Previously using DBA mice it was found that multiple subcutaneous injections of 250 ug/kg of Compound X induced a 3.8-fold increase in CFU-GEMM number per ml of blood compared to the untreated control group and a 3.3-fold increase as compared to the saline control group.

In db/db mice subcutaneous administration of Compound X failed to mobilize progenitor cells into the peripheral blood, even at doses of Compound X as high as 5 mg/kg (results are not shown).

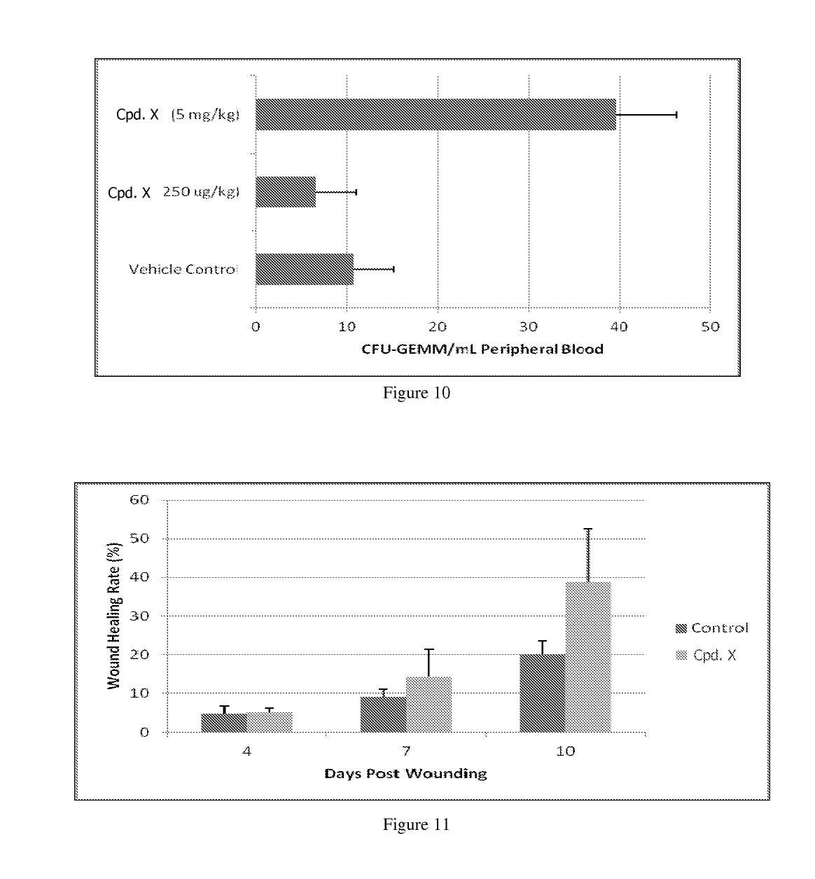

Thus, in the next experiment in db/db mice, 0.9% saline, 250 ug/kg Compound X or 5 mg/kg Compound X were injected intravenously. At one-hour post injection, peripheral blood from 3 mice per group was harvested from the orbital plexus into EDTA-containing tubes. To determine mobilization of colony-forming cells out of the peripheral blood, mononuclear cells were plated into MethoCult 3434 media (Stem Cell Technologies 03434) as before. CFU-GEMM number in the peripheral blood of mice injected intravenously with 5 mg/kg of Compound X was substantially increased (>3 times) compared to mice injected with Vehicle or with the lower dose of Compound X—250 ug/kg (FIG. 10).

Having established an effective i.v. dose of Compound X the effect of progenitor mobilization by Compound X was evaluated in the excisional wound healing model.

The average time to wound closure between db/db mice and non-diabetic C57BL6 mice was compared in a pilot experiment. Paired 6-mm full-thickness cutaneous wounds were made on the dorsa of mice after depilation with a sterile Miltex biopsy punch (VWR #21909-144). To prevent wound contraction during the healing process, 0.5 g/mL polyisobutyl methacrylate (Sigma #445754) in toluene (Sigma #244511) was applied over wounds 24 hours after wounding. After methacrylate hardened, a transparent dressing (Tagaderm, Lab Safety Supply #53379) was placed over wounds. Two groups of mice (5 mice per group) received intravenous injections of either Vehicle or Compound X (5 mg/kg) starting on day one post wounding and continuing daily for four consecutive days at Zeitgeber time 4 (ZT4), 4 hr after onset of light to time mobilization of stem cells with circadian oscillations (Frenette et. al. 2008). The diabetic mice used in this and the following experiments had significantly elevated serum glucose level compared to aged matched C57Bl6 mice.

Digital photographs of the wounds were taken on days 0, 7, 10 and 14. Wound areas were measured by manually tracing the border of unhealed area and quantified as a percent area of the original wound size. The size of the traced area was calculated automatically using the Image J (NIH) analysis program. The following calculation was used to determine the wound healing rate: (Area of original wound−Area of remaining wound)÷Area of original wound×100.

Preliminary experiments have shown that the average time to wound closure in wild-type DBA animals was 8-10 days, while it took db/db mice an average of 18-21 days.

Intravenous injections of 5 mg/kg of Compound X increased the number of CFU-GEMM in peripheral blood of diabetic db/db mice. Wound healing was also accelerated; ten days after wounding, 39% wound closure was observed in the Compound X group compared to 20% closure in the control group (Table 17; FIG. 11).

TABLE 17
Wound healing rate (%) in mice treated with Cpd. X (6-10) versus Saline
(1-5).

| | Day | | | | | | |
|---|---|---|---|---|---|---|---|
| Mouse | 4 | Day 7 | Day 10 | Mouse | Day 4 | Day 7 | Day 10 |
| 1 | 3.36 | 9.76 | 15.09 | 6 | 2.42 | 7.85 | 65.36 |
| 2 | 1.79 | 8.66 | 20.14 | 7 | 8.09 | 8.83 | 17.51 |
| 3 | 4.76 | 6.93 | 21.44 | 8 | 6.62 | 42.89 | 79.10 |
| 4 | 1.19 | 4.27 | 11.64 | 9 | 3.89 | 5.14 | 17.47 |
| 5 | 12.36 | 15.87 | 32.07 | 10 | 4.69 | 6.40 | 13.96 |
| Average | 4.69 | 9.10 | 20.07 | | 5.14 | 14.22 | 38.68 |
| STD | 2.02 | 1.93 | 3.48 | | 1.00 | 7.19 | 13.88 |

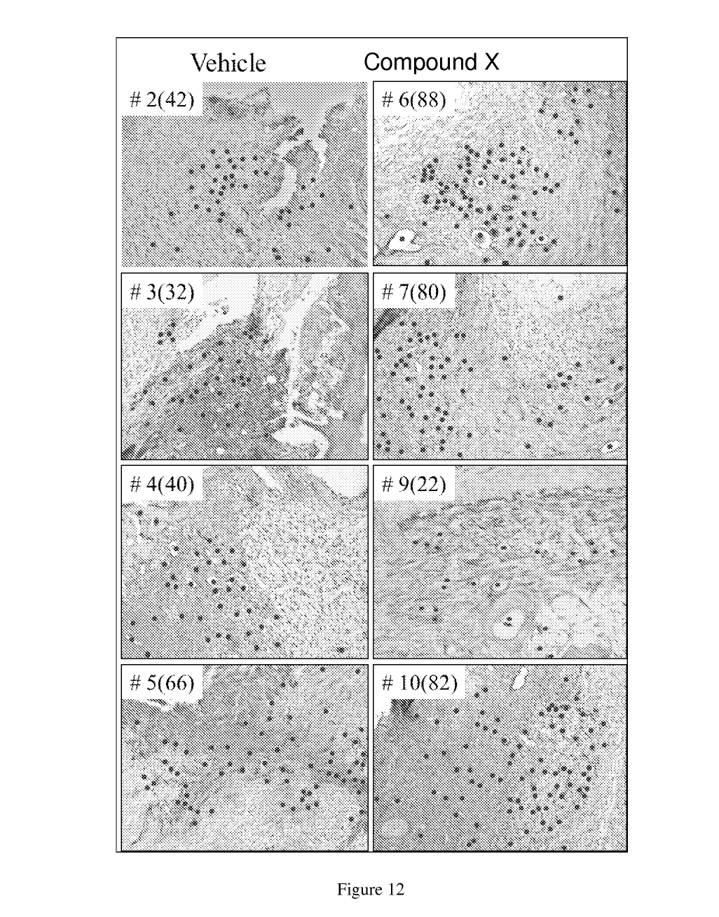

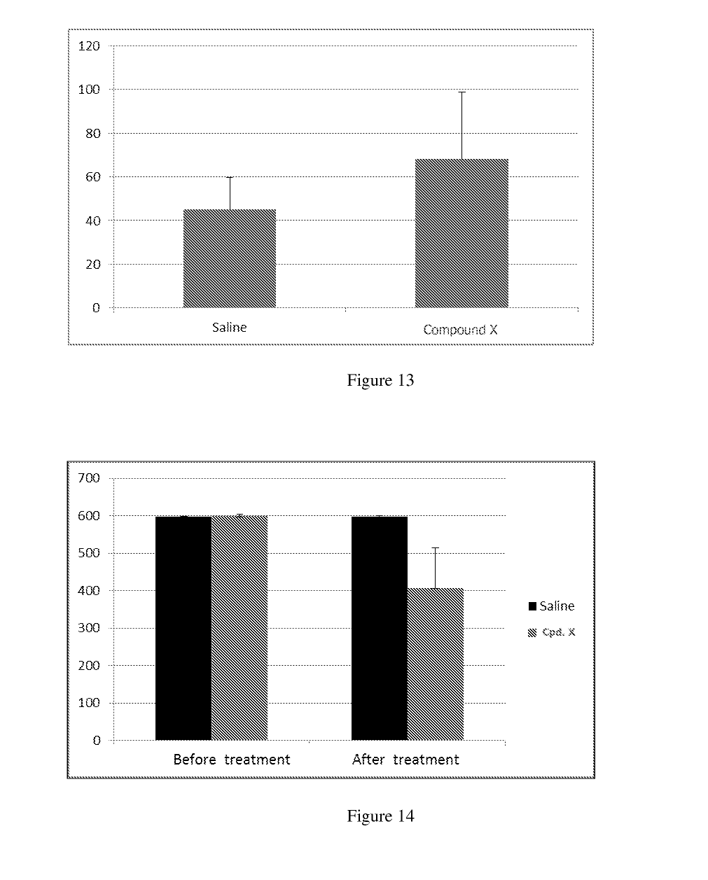

At day 10, wounds were excised, fixed in 4% paraformaldehyde and processed for staining with rabbit anti-α—smooth muscle actin antibodies and vascular density (number of blood vessels counted in the wound dermis area) compared. The number of blood vessels were counted on images as shown on FIG. 12. Results are presented (FIG. 13).

In the next experiment, 2 groups of db/db mice (6 mice per group) received paired 6-mm full-thickness cutaneous wounds. Mice then received intravenous injections of either Vehicle or Compound X (5 mg/kg) starting on day one post wounding and continuing daily for four consecutive days at ZT4. Wounds were monitored until complete closure in control group on day 21. To limit wound contraction during the healing process, a donut shaped silicone splint was centered on the wound and glued (cyanoacrylate adhesive) to the skin. Comparative analysis of the wound area measurements (Image J) was performed at day 14 post-wounding and is presented in Table 18.

TABLE 18
Wound area measurements 14 days post-wounding

| Wound | | | Percent of | Wound | | Day | Percent |
|---|---|---|---|---|---|---|---|
| Saline | Day 0 | Day 14 | original size | Cpd. X | Day 0 | 14 | of original size |
| 1L | 450966 | 107136 | 23.76 | 7L | 522848 | 74676 | 14.28 |
| 1R | 524568 | 179995 | 34.31 | 7R | 394456 | 52782 | 13.38 |
| 2L | 452828 | 139734 | 30.86 | 8L | 446462 | 11094 | 2.48 |
| 2R | 496763 | 115061 | 23.16 | 8R | 394308 | 58870 | 14.93 |
| 3L | 441223 | 57490 | 13.03 | 9L | 527112 | 5865 | 1.11 |
| 3R | 442317 | 53215 | 12.03 | 9R | 369164 | 19152 | 5.19 |
| 4L | 567475 | 54386 | 9.58 | 10L | 497158 | 50090 | 10.08 |
| 4R | 399722 | 30349 | 7.59 | 10R | 302146 | 21199 | 7.02 |
| 5L | 460431 | 73454 | 15.95 | 11L | 472037 | 49486 | 10.48 |
| 5R | 371393 | 50946 | 13.72 | 11R | 439597 | 3603 | 0.82 |
| 6L | 541832 | 139173 | 25.69 | 12L | 560448 | 24952 | 4.45 |
| 6R | 488859 | 128943 | 26.38 | 12R | 472994 | 59032 | 12.48 |
| Average | | | 19.67 | | | | 8.05 |

Note, that mice were taken for the wound healing experiment when their glucose level was high; baseline glucose was measured by glucometer from tail bleed one day before the wound healing experiment next glucose measurement was taken at the end of the study, 7 weeks after the treatment with intravenous injections of Compound X or Saline. As shown (FIG. 14), 4 days treatment course with stem cell mobilizing agent Compound X not only accelerated wound healing process, but decreased glucose level in mice.

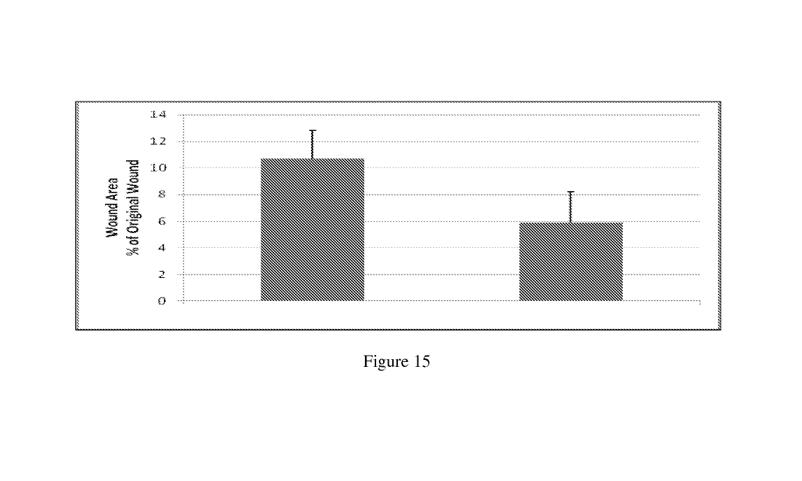

In the next experiment, db/db mice injected i.v. with 5 mg/kg Compound X demonstrated substantially accelerated wound healing. Ten mice were treated per group with 3 mice from each group being sacrificed for histology at day 10 post-wounding. Wound closure in the Compound X treated group was mostly achieved by the 14^{th} day, while control wounds were closed between days 18-21 post-wounding. Wound measurements are presented in Table 6 show that wounds were closed by day 14 in 11 of 14 mice treated with Compound X (95%-100% closure) while mice treated with Saline had only 2 of 14 wounds closed at that time (Table 19; FIG. 15). This was similar to data generated in previous experiments.

TABLE 19
Wound area measurements 14 days post-wounding.

| Wound | | | Percent | Wound | | | Percent of |
|---|---|---|---|---|---|---|---|
| Saline | Day 0 | Day 14 | of original size | Cpd. X | Day 0 | Day 14 | original size |
| 4R | 496860 | 76833 | 15.46 | 14L | 521607 | 53715 | 10.3 |
| 4L | 461358 | 57255 | 12.41 | 14R | 441684 | 22554 | 5.11 |
| 5L | 481228 | 57249 | 11.90 | 15L | 389340 | 20406 | 5.24 |
| 5R | 491856 | 84630 | 17.21 | 15R | 565560 | 168663 | 29.82 |
| 6L | 454518 | 40191 | 8.84 | 16L | 492021 | 4104 | 0.83 |
| 6R | 488370 | 45783 | 9.37 | 16R | 446175 | 24930 | 5.59 |
| 7L | 468508 | 38580 | 8.23 | 17L | 505089 | 972 | 0.19 |
| 7R | 415029 | 137103 | 33.03 | 17R | 532098 | 100098 | 18.81 |
| 8L | 489912 | 43737 | 8.93 | 18L | 478017 | 7440 | 1.56 |
| 8R | 449853 | 30768 | 6.84 | 18R | 458232 | 2808 | 0.61 |
| 9L | 470457 | 2736 | 0.58 | 19L | 535416 | 324 | 0.06 |
| 9R | 424946 | 34215 | 8.05 | 19R | 459939 | 78 | 0.02 |
| 10L | 451041 | 201 | 0.04 | 20L | 556832 | 180 | 0.03 |
| 10R | 497271 | 40737 | 8.19 | 20R | 534059 | 21924 | 4.11 |

Impaired wound healing is a major cause of increased morbidity and mortality in diabetic patients; the activity of Compound X in accelerating wound healing in diabetic mice supports its use in human wound healing, either alone or incombination with active local wound treatments including but not limited to PDGF and other growth factors, or grafts of natural or synthetic (cell-based) skin substitutes.

The reduction in blood glucose observed after treatment with intravenous Compound X indicates that mobilization of circulating progenitor cells supports pancreatic islet preservation or recovery in db/db mice, in addition to supporting wound healing. Mobilization of bone-marrow-derived repair cells that maintain or support regeneration of pancreatic islets with Compound X is a new modality for treatment and prevention of diabetes.

### Example 8

### Multiple Intravenous Injections of Compound X in Diabetic db/db Mice Decreased the Level of Blood Glucose

A study was conducted to determine whether treatment with Compound X could reduce the onset or severity of progressive severe type 2 diabetes using the db/db mouse model, which exhibits rapid onset of islet failure after an initial period of insulin resistance.

Homozygous Leptin receptor-deficient diabetic db/db male mice—BKS.Cg-Dock7m+/+Leprdb/J (Jackson Laboratories, stock #000642) become obese at 3-4 weeks of age. Elevation of plasma insulin usually occurs at 10-14 days and elevation of blood glucose at 6-8 weeks of age. We examined the effects of Compound X on the onset of hyperglycemia in db/db mice. Continuous Compound X treatments were performed during 5 weeks.

At day 0, homozygous 7-week-old female db/db mice were divided into 2 experimental groups (n=10); both groups received subcutaneous injections for 5 consecutive days: Group 1 received 0.9% saline and group 2 received 5 mg/kg Compound X. The following week mice received intravenous injections for 4 consecutive days. The next two weeks mice received subcutaneous injections 3 times per week and the last week of treatment was given intravenously 3 times per week.

Nonfasting blood glucose levels were measured weekly from tail bleeds with a handheld glucometer. Animal body weights were taken throughout the course of the study.

At the end of experiment, serum from blood was obtained from the retro-orbital sinus after an overnight fast.

The dynamics of hyperglycemia development in db/db mice are presented in Table 20. As shown by weekly measurements of nonfasting glucose level in blood, a sharp increase in glucose level started at week 8 of age; at all timepoints, the group of mice treated with Compound X had a decreased glucose level as compared to the control group of mice treated with Saline.

TABLE 20
Glucose level in peripheral blood of db/db mice treated for 5 weeks

| | | Week 1 | Week 2 | Week 3 | Week 4 | Week 5 | Week 6 |
|---|---|---|---|---|---|---|---|
| Control | 1 | 173 | 546 | HI | HI | HI | HI |
| | 2 | 448 | 581 | 594 | 572 | HI | HI |
| | 3 | 149 | 370 | 422 | 456 | 491 | HI |
| | 4 | 177 | 492 | HI | HI | HI | HI |
| | 5 | 180 | 434 | HI | HI | HI | HI |
| | 6 | 221 | 475 | 552 | 558 | 469 | HI |
| | 7 | 150 | 427 | 369 | 495 | 454 | HI |
| | 8 | 169 | 518 | HI | HI | HI | HI |
| | 9 | 297 | 567 | HI | HI | 592 | HI |
| | 10 | 174 | 473 | 514 | 594 | 441 | HI |
| | | 214 ± 29 | 488 ± 21 | 545 ± 27 | 568 ± 16 | 545 ± 22 | 600 ± 0 |
| Compound X | 11 | 160 | 481 | 541 | 398 | 481 | 400 |
| | 12 | 121 | 377 | 447 | 416 | 443 | 541 |
| | 13 | 122 | 177 | 271 | 206 | 256 | 265 |
| | 14 | 235 | 472 | HI | 599 | 527 | 537 |
| | 15 | 117 | 273 | 546 | 440 | 525 | 576 |
| | 16 | 178 | 452 | 496 | 458 | 582 | HI |
| | 17 | 248 | 500 | 474 | 591 | 588 | HI |
| | 18 | 119 | 189 | 267 | 257 | 467 | 457 |
| | 19 | 181 | 502 | 541 | HI | HI | HI |
| | 20 | 194 | 430 | 463 | 549 | HI | HI |
| | | 168 ± 15 | 385 ± 40 | 465 ± 36 | 451 ± 44 | 506 ± 33 | 517 ± 35 |
| | | P = .385 | *P = .035 | P = .055 | *P = .022 | P = .33 | *P = .006 |

(High/Hi = ≧600 mg/dL, the maximum possible glucometer reading; high values were set to 600 mg/dL as a conservative estimate)
*= significantly lower than vehicle control by Mann-Whitney test

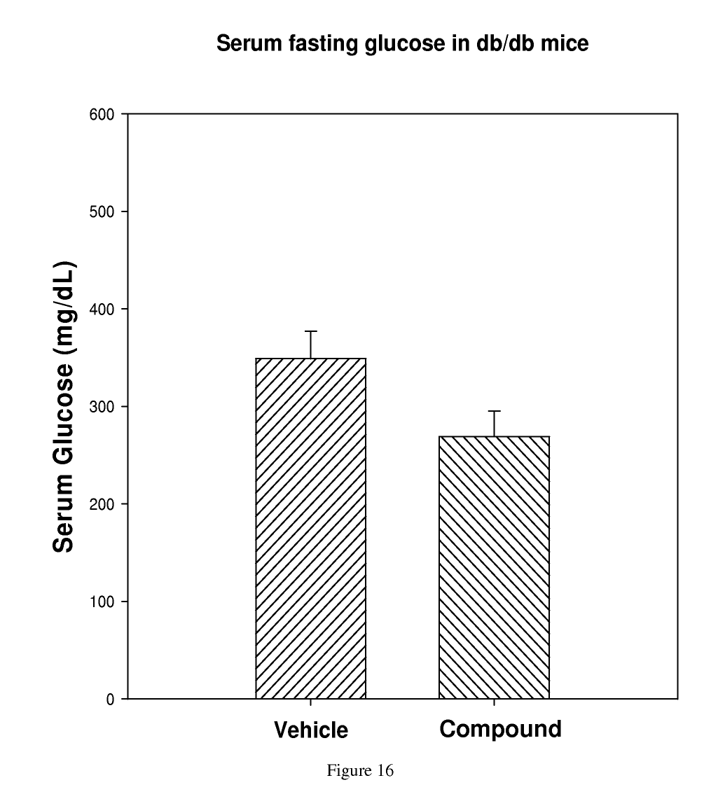

Overnight fasting serum glucose measurements from blood samples taken at the end of the 5-week study were performed by AniLytics Inc. (Gaithersburg, Md.); showing that in the control group treated with Vehicle only, 3/10 mice had fasting glucose below 300 mg/dL, while in the Compound X-treated group 7/10 mice had a glucose level <300 mg/dL and 2 mice had lower than 200 mg/dL serum glucose, with mean serum fasting glucose values for groups of mice treated with vehicle versus Compound X shown in FIG. 16.

The attenuation of hyperglycemic progression in db/db mice by Compound X is believed to be due to islet preservation due to mobilization of supportive progenitor cells from the bone marrow. This represents a new approach to prevention and treatment of type 2 diabetes, complementary to other antihyperglycemic and islet-preserving pharmacologic therapies.

### LITERATURE REFERENCES

1. Broxmeyer, H. E., C. M. Orschell, D. W. Clapp, G. Hangoc, S. Cooper, P. A. Plett, W. C. Liles, X. Li, B. Graham-Evans, T. B. Campbell, G. Calandra, G. Bridger, D. C. Dale and E. F. Srour 2005. “Rapid mobilization of murine and human hematopoietic stem and progenitor cells with AMD3100, a CXCR4 antagonist.” J Exp Med 201(8): 1307-18.
2. Broxmeyer, H. E., G. Hangoc, S. Cooper, T. Campbell, S. Ito and C. Mantel 2007. “AMD3100 and CD26 modulate mobilization, engraftment, and survival of hematopoietic stem and progenitor cells mediated by the SDF-1/CXCL12-CXCR4 axis.” Ann N Y Acad Sci 1106: 1-19.
3. Christopherson, K. W., 2nd, S. Cooper and H. E. Broxmeyer (2003). “Cell surface peptidase CD26/DPPIV mediates G-CSF mobilization of mouse progenitor cells.” Blood 101(12): 4680-6.
4. Christopherson, K. W., 2nd, G. Hangoc and H. E. Broxmeyer (2002). “Cell surface peptidase CD26/dipeptidylpeptidase IV regulates CXCL12/stromal cell-derived factor-1 alpha-mediated chemotaxis of human cord blood CD34+ progenitor cells.” J Immunol 169(12): 7000-8.
5. De Meester, I., S. Korom, J. Van Damme and S. Scharpe (1999). “CD26, let it cut or cut it down.” Immunol Today 20(8): 367-75.
6. Ferraro F, Lymperi S, Mendez-Ferrer S, et al. Diabetes impairs hematopoietic stem cell mobilization by altering niche function. Sci Transl Med 2011; 3(104).
7. Paolo Fiorina, Giorgio Pietramaggiori, Saja S. Scherer, Mollie Jurewicz, Jasmine C. Mathews, Andrea Vergani, Gebhard Thomas, Elena Orsenigo, Carlo Staudacher, Stefano La Rosa, Carlo Capella, Adelaide Carothers, Hans Gunter Zerwes, Livio Luzi, Reza Abdi, and Dennis P Orgill: The Mobilization and Effect of Endogenous Bone Marrow Progenitor Cells in Diabetic Wound Healing (2010) Cell Transplantation v19 pp 1369-1381.
8. Kareem A, Nervi B, Ramirez P, Rettig M P, et al. 2009. “Chemosensitization of AML following mobilization by the CXCR4 antagonist AMD3100.” Blood; 113:6206-6214).
9. Krause D S, Theise N D, Collector M I, Henegariu O, Hwang S, Gardner R, Neutzel S, Sharkis S J. 2001. “Multi-organ, multi-lineage engraftment by a single bone marrow-derived stem cell”. Cell; 105:369-377.
10. Lapidot, T., A. Dar and O. Kollet. 2005; “How do stem cells find their way home?” Blood 106(6): 1901-10.
11. Lapidot, T. and I. Petit. 2002; “Current understanding of stem cell mobilization: the roles of chemokines, proteolytic enzymes, adhesion molecules, cytokines, and stromal cells.” Exp Hematol 30(9): 973-81.
12. Lucas, D., Battista M., Shi A., Isola L. Frennette P. 2008 “Mobilized Hematopoietic stem cell yield depends on species-specific circadian timing”. Cell Stem Cell; 3: 364-366.
13. Mendez-Ferrer S, Chow A, Merad M, Frenette P S. Circadian rhythms influence hematopoietic stem cells. (2009) Curr Opin Hematol. July 16(4):235-42.
14. Nervi, B., D. C. Link and J. F. DiPersio 2006; “Cytokines and hematopoietic stem cell mobilization.” J Cell Biochem 99(3): 690-705.
15. Orlic D, Kajstura J, Chimenti S, Limana F, Jakoniuk I, Quaini F, Nadal-Ginard B, Bodine D M, Leri A, Anversa P. 2001. Mobilized bone marrow cells repair the infarcted heart, improving function and survival. Proc Natl Acad Sci USA; 98:10344-10349.
16. Peled, A., I. Petit, O. Kollet, M. Magid, T. Ponomaryov, T. Byk, A. Nagler, H. Ben-Hur, A. Many, L. Shultz, O. Lider, R. Alon, D. Zipori and T. Lapidot 1999. “Dependence of human stem cell engraftment and repopulation of NOD/SCID mice on CXCR4.” Science 283(5403): 845-8.
17. Pelus, L. M. and S. Fukuda 2008. “Chemokine-mobilized adult stem cells; defining a better hematopoietic graft.” Leukemia 22(3): 466-73.
18. Rafii S, Lyden D. 2003. “Therapeutic stem and progenitor cell transplantation for organ vascularization and regeneration”. Nat Med; 9:702-712.
19. Rosu-Myles, M., L. Gallacher, B. Murdoch, D. A. Hess, M. Keeney, D. Kelvin, L. Dale, S. Ferguson, D. Wu, F. Fellows and M. Bhatia 2000. “The human hematopoietic stem cell compartment is heterogeneous for CXCR4 expression.” Proc Natl Acad Sci USA, 97(26): 14626-31.
20. Shizuru, J. A., R. S, Negrin and I. L. Weissman 2005. “Hematopoietic stem and progenitor cells: clinical and preclinical regeneration of the hematolymphoid system.” Annu Rev Med 56: 509-38.
21. Singer, A. J.; Clark, R. A. Cutaneous wound healing. N.; 1349-1353; 1981. Engl. J. Med. 341(10):738-746; 1999)
22. Tavor S, Petit I., Porozov S, Goichberg P, Avigdor A., Sagiv S., Nagler A., Naparstek E, Lapidot T. 2005. “Motility, proliferation, and egress to the circulation of human AML cells are elastase dependent in NOD/SCID chimeric mice” Blood, 106: 2120-2127).
23. Oren M. Tepper, Jacquelyn Carr, Robert J. Allen Jr., Christopher C. Chang, Clarence D. Lin, Rica Tanaka, Sanjeev M. Gupta, Jamie P. Levine, Pierre B. Saadeh, and Stephen M. Warren Decreased Circulating Progenitor Cell Number and Failed Mechanisms of Stromal Cell-Derived Factor-1 Mediated Bone Marrow Mobilization Impair Diabetic Tissue Repair (2010) Diabetes v59 pp 1974-1983;

1. A method of mobilizing stem cells from bone marrow of a subject, comprising administering to the subject an amount of (SEQ ID NO: 1) effective to mobilize the stem cells.

2-4. (canceled)

5. The method of claim 1, wherein the subject is a human and the amount of (SEQ ID NO: 1) is from about 100 micrograms to about 10 milligrams per administration, administered from 1 time per week to 3 times per day.

6. A method for treating a subject in need of one or more of preservation, repair, or regeneration of bodily tissue, or revascularization, comprising the method of claim 1, thereby promoting the one or more of preservation, repair, or regeneration of bodily tissue or regeneration in the subject.

7. A method for treating a subject in need of one or more of preservation, repair, or regeneration of bodily tissue, or revascularization, comprising the method of claim 5, thereby promoting the one or more of preservation, repair, or regeneration of bodily tissue or regeneration in the subject.

8. The method of claim 6, wherein the tissue that is preserved, repaired or regenerated is pancreatic tissue.

9. A method of treating diabetes comprising the method of claim 8.

10. The method of claim 7, wherein the tissue that is preserved, repaired or regenerated is pancreatic tissue.

11. A method for treating diabetes comprising the method of claim 10.

12. The method of claim 6, wherein the tissue that is preserved, repaired, or regenerated is dermal tissue.

13. A method for treating a dermal wound, comprising the method of claim 12.

14. The method of claim 7, wherein the tissue that is preserved, repaired, or regenerated is dermal tissue.

15. A method of treating a dermal wound, comprising the method of claim 14.
